(19) United States
(12) Patent Application Publication
(10) Pub. No.: US 2013/0196944 A1
(43) Pub. Date: Aug. 1, 2013

(54) FILLER COMPOSITION COMPRISING BETA-GLUCANS

(71) Applicant: Heiko Barg, Weinheim (DE)

(72) Inventor: Heiko Barg, Weinheim (DE)

(73) Assignee: MERZ PHARMA GmbH & CO. KGaA, Frankfurt am Main (DE)

(21) Appl. No.: 13/816,585

(22) PCT Filed: Aug. 17, 2011
(86) PCT No.: PCT/EP2011/004146

§ 371 Date: Apr. 11, 2013

Related U.S. Application Data
(60) Provisional application No. 61/375,116, filed on Aug. 19, 2010.

(30) Foreign Application Priority Data
Aug. 19, 2010 (EP) 10008678.4

(51) Int. Cl. A61L 27/20

(52) U.S. Cl. CPC A61L 27/20
USPC 514/54; 536/120

(57) ABSTRACT

The present invention pertains to a filler composition comprising β-glucan moieties and optionally a cosmetically and/or pharmaceutically acceptable carrier. It further relates to a filler composition, wherein the β-glucan moieties are cross-linked. in one embodiment of the instant invention the filler composition is a dermal filler. In one further embodiment of the present invention the filler composition is for the treatment of wrinkles and/or folds. In another embodiment of the instant invention the filler composition is for use in the treatment of a medical condition. The filler composition provided in the present invention may further comprise one or more active pharmaceutical ingredients. Further, the present invention pertains to a process for preparing the filler composition as claimed herein.

### FIELD OF THE INVENTION

The present invention pertains to a filler composition comprising β-glucan moieties and optionally a cosmetically and/or pharmaceutically acceptable carrier. It further relates to a filler composition, wherein said β-glucan moieties are cross-linked. In one embodiment of the instant invention the filler composition is a dermal filler. In one further embodiment of the present invention the filler composition is for the treatment of wrinkles and/or folds. In another embodiment of the instant invention the filler composition is for use in the treatment of a medical condition. The filler composition provided in the present invention may further comprise one or more active pharmaceutical ingredients. Further, the present invention pertains to a process for preparing the filler composition as claimed herein.

### BACKGROUND OF THE INVENTION

Treatment with fillers is known since the 1980s. Today's most preferred fillers can be classified as hyaluronic acid-based fillers (Hylaform®, Hylaform® Plus, Restylane®, Perlane®, Juvederm®, Juvederm® Ultra, Juvederm® Ultra Plus, Puragen®, Puragen® Plus, Matridur®), polymethylmethacrylate (PMMA) based fillers (Artecoll®, Artefill®, Artesense™), collagen based fillers (Zyderm® I, Zyderm® II, Zyplast®, Atelocollagen®, CosmoDerm® I, CosmoDerm® II, Resoplast®) and alginate based fillers (e.g. Novabel®) as described in DE 10 2004 019 241.

These different fillers are used for tissue augmentation or as implants in plastic and esthetic surgery. Filler compositions based on, for example, cross-linked PMMA often show volume-spending properties. When used for tissue augmentation or as an implant, such PMMA-based fillers may have negative impact on the tissue of the application, i.e. cause inflammatory reactions or formation of granulomas. At best they do not have any influence on the tissue they were applied to.

Pharmaceutical ingredients like antioxidants or inflammation inhibitors were added to filler compositions to generate a positive effect of the filler to the treated tissue. Due to their metabolism and distribution into adjacent tissues these pharmaceutical ingredients only remain briefly in the area of application. The positive effects decrease rapidly.

Collagen is a natural protein of connective tissue. However, some people suffer from allergic reactions to collagen and thus, an allergy test is always suggested by the practitioner prior to injection of fillers comprising collagen. Hyaluronic acid is a polysaccharide and is naturally found in many tissues of the body. The unfavorable effect of fillers comprising hyaluronic acid is the short-lasting result and the need for multiple injections for an observable effect. Thereby swellings can occur, which decay in 1-3 days. Thus, treatments with collagen and hyaluronic acid based fillers are costly and painful due to the prerequisite of multiple injections and allergy tests. Further reported complications for the fillers is poor syringeability due to high viscosity, aggregation of the particles in the packaging and non-homogeneous distribution of the particles at the injection site.

The use of alginate as filler is known from DE 10 2004 019 241. However, DE 10 2004 019 241 suggests for the long-lasting effect of the cross-linked alginate particles the use of barium.

U.S. Pat No. 6,544,503 B1 relates a process for the preparation of cross-linked water-swellable polymer particles as well as a method of soft tissue augmentation comprising administering to a patient an aqueous dispersion of cross-linked water-swellable polymer particles in an amount to effectively augment the affected tissue of the patient. The disclosed particles are substantially homogeneous in size showing a diameter between about 10 μm and about 250 μm and wherein at least 80% of the particles are spherical.

WO 2008/103594 describes the use of chitosan and its derivatives as biomaterial for the treatment, repair and/or enhancement of bodily tissue insufficiencies of the vocal chords, muscles, ligaments and cartilage. According to WO 2008/103594 the use of the biomaterial produces a filling effect. Also disclosed are chitosan or chitosan-derivative gels, which optionally include chitin microspheres. In a first step of the chitin microsphere preparation, chitosan microspheres were obtained by spray-drying a chitosan solution in acetic acid/ethanol. WO 2008/103594 does not describe gels of cross-linked chitosan.

EP 0 830 416 (equivalent of U.S. Pat. No. 6,214,331) describes the preparation of a cross-linked water-soluble polymer particle preparation, wherein the particles are less than 212 μm in diameter and wherein at least 80% of the particles are spherical, obtainable by adding an aqueous polymer solution, comprising a water-soluble polymer β-glucan and an aqueous medium, to an oil base containing a water in oil emulsifying agent, agitating the mixture to form an emulsion containing polymer droplets, and cross-linking the polymer droplets in situ by a cross-linking agent resulting in the formation of cross-linked polymer particles. For the production of hyaluronic acid microspheres the cross-linking agent is added directly to an emulsion of aqueous hyaluronic acid in toluene. The cross-linking agent is first deactivated by adjusting the pH of the aqueous solution to pH 11 and then activated by lowering the pH to 7 to 8. It is preferred to use toluene, o-xylene or isooctane as oil phase. The weight ratio of aqueous phase to oil phase is about 1 to 1. EP 0 830 416 does not describe monophasic gels.

WO 2010/056899 describes the preparation of gels based on cellulose derivatives, which are obtained by cross-linking carboxymethyl cellulose functionalized with photo- or redox-crosslinkable groups. WO 2010/056899 does not describe gels obtained by chemical cross-linking.

Therefore, there remains a continuous need for an improved filler composition with an improved safety profile, an improved tolerability profile, improved application characteristics and a long-lasting effect.

### OBJECTS OF THE INVENTION

Accordingly, in view of the problems of the prior art, a first object of the present invention is to provide a filler composition with a positive effect on the tissue surrounding the application site. Thereby it should be the volume-spending compound itself having positive influence on the tissue.

The second object of the present invention is a novel filler composition, which further comprises one or more active pharmaceutical ingredients, particularly for sustained release of said one or more active pharmaceutical ingredients.

The third object of the present invention is a novel filler composition for injection, rapidly restoring volume at the application site and sustaining the volume augmentation, and which does not contain collagen, thereby not requiring pre-testing for allergic reactions. It is also important that the filler composition remains evenly distributed after the injection to avoid palpable mass after the carrier is resorbed in the body. Thus, it is an object of the present invention to provide a novel filler composition exhibiting a long-lasting effect and far less side effects.

Another object of the present invention is to provide a novel filler composition, which, unlike conventional fillers, does not contain collagen, polymethylmethacrylate or hyaluronic acid or derivatives thereof as a major component, is not easily degraded by human enzymes or absorbed in the body, thus ensuring stable longer-lasting volume augmentation, and is cheaper than conventional fillers.

One further object of the instant invention is to provide a filler composition exhibiting a more improved syringeability as the conventional fillers, avoidance of aggregation of the particles in the packaging and non-homogeneous distribution of the particles at the injection site.

### SUMMARY OF THE INVENTION

These and other objects are solved by a filler composition comprising β-glucan moieties and optionally a cosmetically and/or pharmaceutically acceptable carrier.

In one embodiment of the instant invention said β-glucan moieties are selected from branched and unbranched β-glucan moieties, particularly β-(1,3)-glucan, β-(1,4)-glucan, β-(1,3)-(1,4)-glucan, β-(1,3)-(1,6)-glucan or β-(1,4)-(1,6)-glucan moieties. More particularly, said β-glucan moieties are selected from the group of: cellulose, chitin, chitosan, curdlan, laminarin, chrysolaminarin, lentinan, lichenin, pleuran, zymosan, schizophyllan and scleroglucan, β-glucans extracted from grain, in particular oat or barley, and any β-glucans produced by biotechnological means. In certain embodiments, the β-glucan moieties are a mixture of different β-glucan moieties, each selected from the group listed above. The β-glucan moieties according to the present invention are β-glucan moieties acceptable for dermal topical application and for injection into soft tissue.

In particular embodiments of the present invention, the β-glucan moieties are cross-linked.

In particular embodiments, the β-glucan moieties do not comprise moieties that are photo- or redox-crosslinkable.

The cross-linked β-glucan moieties can be obtained according to another embodiment of the present invention by reacting β-glucan moieties with a bifunctional cross-linking agent, particularly divinyl sulfone (DVS), butanediol diglycidyl ether (BDDE), 1,2,7,8-diepoxyoctane (DEO), 1,2,3,4-diepoxybutane, polyethylene glycol diglycidyl ether or with a multifunctional cross-linking agent, particularly polyethylene glycol epoxide.

In one embodiment of the instant invention, the filler composition does not contain substantial amounts of hyaluronic acid, polymethylmethacrylate, collagen or derivatives thereof.

In another preferred embodiment, the filler composition further comprises one or more active pharmaceutical ingredients, particularly wherein said one or more active pharmaceutical ingredients are linked to said β-glucan moieties or said bifunctional cross-linking agent. These ingredients may be selected from the group of anesthetics, local anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents, biorejunevation agents, anti-aging agents, anti-bacterial agents, anti-histaminic agents, healing supporting agents, anti-acne agents, moisturizing agents, tanning agents and/or anti-cellulite agents.

The filler composition may further comprise one or more cosmetically and/or pharmaceutically acceptable carrier selected from the group containing high purity water, glycerol, phosphate buffered saline, carboxy-methylcellulose, and hyaluronic acid, or a mixture thereof. Additionally, it may contain one or more pharmaceutical excipients selected from antioxidants, viscosity enhancers and/or modifiers, hydrating agents, bulking substances, tonicity agents, preservatives and surface active agents, or a mixture thereof.

In particular embodiments, the filler composition forms a gel. This gel may have thixotropic viscosity properties.

In one embodiment of the instant invention the concentration of the β-glucan moieties is between about 0.1 and about (i) 5.0 wt.-% for β-glucans>100 kDa, or (ii) 10.0 wt.-% for β-glucans<=100 kDa, in each case of the final total weight of filler composition.

In a particular embodiment the cross-linked β-glucan moieties forms a monophasic gel

In another embodiment the gel forms particles, particularly wherein the gel particles are elastic and approximately spherical. In particular embodiments, the particles have mass median diameter of between about 60 and about 200 μm determined by microscopical analysis.

In another embodiment of the present invention, the β-glucan or the cross-linked β-glucan has a mean molecular weight from about 1 kDa to about 3 mDa.

The filler composition of the present invention particularly is an injectable filler composition.

In one embodiment of the instant invention the filler composition is for use for aesthetic purposes.

In one further embodiment of the present invention, the filler composition is for use as a dermal filler.

In one embodiment of the present invention, the dermal filler is for the treatment of, or for the use in the treatment of, wrinkles and/or folds.

In another embodiment of the present invention, the filler composition is for the treatment of, or for the use in the treatment of, a medical condition, including lipoatrophy, gastroesophageal reflux disease (GERD), urine incontinence, vesico-ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles, breast and acne scars.

In another aspect, the present invention relates to the use of the filler composition for the treatment of, or for the use in the treatment of, acne scars, such as by filling areas of acne scars.

In another aspect, the present invention relates to the use of the filler composition for the treatment of, or for the use in the treatment of, a medical condition, including lipoatrophy, gastroesophageal reflux disease (GERD), urine incontinence, vesico-ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a filler composition as claimed in the present invention to a patient in need thereof.

In another aspect, the present invention relates to a method for treating one of the conditions or diseases mentioned hereinabove.

The present invention further pertains to a method of using a filler composition according to the present invention in plastic, cosmetic, dental or general surgery, in ophthalmology, in orthopedics, as products for preventing tissue adhesions, or in urology, wherein said method comprises a step of administering a filler composition as claimed in the present invention to a patient in need thereof.

Further, the present invention pertains to a process (i.e. a method, such as a manufacturing method) for preparing the filler composition as claimed herein, wherein the process comprises a step of combining a solution of at least one β-glucan moiety with an aqueous solution containing at least one cross-linking agent or combining a solution of at least one β-glucan moiety with an aqueous solution containing at least one cross-linking agent.

In particular embodiments of the present invention, said process comprises the step of adding a crosslinking reagent, particularly BDDE, to a concentrated non-crosslinked beta-glucan gel.

Further, the present invention pertains to a process (i.e. a method, such as a manufacturing method) for preparing the filler composition as claimed herein, wherein the process comprises a step of combining an aqueous solution of the β-glucan moieties and a hydrophobic medium containing a water-in-oil emulsifier so as to form an emulsion of droplets of the β-glucan moieties; and adding to the emulsion a cross-linking agent capable of cross-linking the β-glucan moieties.

In further embodiments, said process comprises the step of combining a solution of at least one β-glucan moiety with an aqueous solution containing at least one cross-linking agent, particularly wherein:

a. the pH of the aqueous solution containing at least one cross-linking agent is adjusted to a pH value between about 6 and about 10, particularly between about 6.5 and about 8.5;

b. the concentration of the cross-linking agent is between about 2.5 wt-% (w/w β-glucan) and 30 wt-% (w/w β-glucan), particularly between about 2.5 wt-% (w/w β-glucan) and 15 wt-% (w/w β-glucan);

c. the concentration of β-glucan moieties is between about 0.1 and about (i) 5.0 wt.-% for β-glucans>100 kDa, or (ii) 10.0 wt.-% for β-glucans<=100 kDa, in each case of the final total weight of filler composition;

d. the viscosity of the solution of β-glucan moieties is adjusted in a way that a force of between about 15 N and about 50 N is required for pressing the solution of β-glucan moieties through a 27G1/2 needle; and/or

e. said aqueous solution containing the cross-linking agent further comprises one or more active pharmaceutical ingredients selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

Further, the present invention pertains to a kit comprising a filler compound as provided herein and an injection device. The injection device could be a syringe or an electronic injection device.

Further, the present invention pertains to an injection device comprising the filler composition provided herein. The injection device could be a prefilled syringe or an electronic injection device.

### DETAILED DESCRIPTION OF THE INVENTION

The present invention relates to a filler composition comprising β-glucan moieties and optionally a cosmetically and/or pharmaceutically acceptable carrier.

The terms “comprise” and “contain” within the meaning of the invention introduce a non exhaustive list of features. Likewise, the word “one” is to be understood in the sense of “at least one”.

The term “filler composition” as used herein relates to compositions administered for augmentation, repair or strengthening of tissue or for filling a bodily cavity in a human or animal taken from the list of farm animals like horse, cattle, pig, camel, chicken, turkey or pets like dog or cat. The volume augmentation in this respect is a long lasting increase of volume by virtue of the components forming the filler compositions, where the components do not undergo rapid diffusion

The term “polymer” in the present invention relates to macromolecules composed of repeating structural units connected by chemical bonds.

The term “β-glucan” as used in the present invention relates to polysaccharides of D-glucose monomers linked by β-glycosidic bonds. β-glucans can vary with respect to molecular mass, solubility, viscosity and three-dimensional configuration. Some of the hydroxyl groups of the D-glucose monomers may be substituted by acetylamino, amino, quaternized amino, carboxyl, and/or thiol groups.

An “injectable composition” according to the present invention is a composition that is applied into a human or animal body intramuscular, intradermal/intracutaneous or subcutaneous or as an implant. An injectable composition has to be sterile and non-pyrogenic. It should be as isosmotic and as euhydric as possible.

The term “mean molecular weight” relates to the molecular weight at the maximum of the distribution of the molecular weights of a population of polymer molecules (β-glucan molecules) if the molecular weight follows a Gaussian distribution. The polymer molecules are of different branching and length and therefore not of identical size and weight. Thus, the population could be best characterized with mean value for the molecular weight the β-glucan molecules and the standard deviation.

The term “active pharmaceutical ingredient” refers to all structures, which are pharmacologically active, thus resulting in a pharmacological effect in mammal and all known chemical forms thereof. Examples are, but not limited to, conjugates, isomers, esters, derivatives, metabolites, residues, salts or prodrugs thereof.

The term “carrier” is used in the present invention to describe a solvent or suspension medium for the β-glucan moieties, which is essentially pharmacologically inactive and essentially without volume-spending or tissue-augmenting properties.

The term “pharmaceutical excipients” refers to structures, which are essentially pharmacologically inactive and essentially without volume-spending or tissue-augmenting properties. Thus, pharmaceutical excipients may include antioxidants, preservatives and the like.

In the instant invention the filler composition contains at least one β-glucan moiety and a cosmetically and/or pharmaceutically carrier. Thus, the at least one β-glucan moiety leads to a long-lasting volume augmentation. Unlike, the carrier functions as a medium to dissolve or suspend the β-glucan moieties. The carrier further could compensate an isomotic and euhydric lack of the β-glucan moieties.

In particular embodiments, the filler composition of the present invention essentially consists of the one or more β-glucan moieties, which could be covalently or non-covalently bonded with one or more active pharmaceutical agents.

The β-glucan is a polysaccharide of D-glucose units particularly linked by (1,3)- or (1,4)-glycosidic bonds or by (1,3)-(1,4)-, (1,3)-(1,6)- or (1,4)-(1,6)-glycosidic bonds. In view of the different linkage it contains branched or unbranched parts of the polysaccharide chain.

Particularly, the β-glucan is selected from the group containing cellulose, chitin, chitosan, curdlan, laminarin, chrysolaminarin, lentinan, lichenin, pleuran, zymosan, schizophyllan, scleroglucan, β-glucans extracted from grain, in particular oat or barley or the same β-glucans produced by biotechnological means. All these β-glucans may be substituted by common substituents like acetylamino, amino, carboxyl, quaternized amino, carboxyl, and/or thiol groups. The β-glucan may be selected i.e. from the group containing chitosan, chitosan glycolate, chitosan lactate, chitosan acetate, chitosan succinate, N-(aminoalkyl) chitosan, succinyl chitosan, quateraminated chitosan, octanoyl chitosan, acetyl chitosan, thiol chitosan, trimethyl chitosan, carboxymethyl chitosan, trimethyammonium chitosan, N-diethyl methyl chitosan, N-methyl chitosan, carboxymethyl chitosan, N-carboxyethyl chitosan, glycol chitosan, N-(2-hydroxy)propyl-3-trimethylammonium chitosan or mixtures thereof.

In a preferred embodiment of the present invention the β-glucan moieties are cross-linked β-glucans. The cross-linked β-glucans are available by reacting a β-glucan with a bifunctional cross-linking agent. Most preferred cross-linking agents are divinyl sulfone (DVS), butanediol diglycidyl ether (BDDE), 1,2,7,8-diepoxyoctane (DEO), 1,2,3,4-diepoxybutane, polyethylene glycol diglycidyl ether, ethylene glycol diglycidyl ether, 1,4-bis(2,3-epoxypropoxy)butane, 1,4-bisglycidyloxybutane, 1,2-bis(2,3-epoxypropoxy)ethylene, or 1-(2,3-epoxypropyl)-2,3-epoxycyclohexane. To reach a higher degree of cross-link a polyfunctional cross-linking agent, particularly polyethylene glycol epoxide, may be used. The degree of cross-link thereby is controllable by the concentration of cross-linking agent, the concentration of β-glucan, the temperature and, depending on the cross-linking agent, the pH of the solution.

In particular embodiments, the β-glucan moieties do not comprise moieties that are photo- or redox-crosslinkable.

Moieties that are photo- or redox-crosslinkable are described, for example, in WO 2010/056899.

In particular embodiments, the filler composition according to the instant invention comprises a mixture of crosslinked and non-crosslinked β-glucan moieties.

In particular embodiments, the filler composition according to the instant invention does not contain substantial amounts of hyaluronic acid, polymethylmethacrylate or collagen, or chemically modified derivatives of these substances. Thus, in such embodiments, the volume-augmenting compound consists of β-glucan and/or its derivatives. In such embodiments, t is still possible that a minor amount of hyaluronic acid, polymethylmethacrylate or collagen or their modified derivatives, such as less than about 10% (w/w β-glucan), particularly less than about 5% (w/w β-glucan), more particularly less than about 1% (w/w β-glucan), is present for other purposes than volume augmentation, i.e. for a preservative, viscosity adapting, osmotic purpose or tissue stimulating effects.

The β-glucan moieties of the claimed filler composition in the instant invention may further be covalently or non-covalently bonded to one or more active pharmaceutical ingredients. These ingredients are selected from the group containing anesthetics, local anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents, anti-aging agents, anti-bacterial agents, anti-histaminic agents, healing supporting agents, anti-acne agents, moisturizing agents, tanning agents, and/or anti-cellulite agents.

Anesthetics may be, but are not limited to, local anesthetics based on esters (Procaine, Benzocaine, Chloroprocaine, Cocaine, Cyclomethycaine, Dimethodcaine, Larocaine, Propoxycaine, Proparacaine, Tretracaine) or local anesthetics based on amides (Lidocaine, Articaine, Bupivacaine, Carticaine, Cinchocaine, Etidocaine, Levobupivacaine, Mepivacaine, Piperocaine, Prilocaine, Ropivacaine, Trimecaine). A suitable concentration for the anesthetic is from about 0.01% to 6% based on the total weight of the composition and the agent selected.

Analgesics may be, but are not limited to, paracetamol, ibuprofen, diclofenac, naproxen, aspirin, celecoxib, etoricoxib, lumiracoxib, parecoxib, rofecoxib, valdecoxib, nimesulid.

Antimicrobials may be, but are not limited to, antibiotics (amikacin, gentamycin, neomycin, tobramycin, kanamycin, meropenem, imipenem, cefaclor), antivirals (abacavir, aciclovir, amantadine, boceprevir, cidofovir, darunavir, edoxudine, famciclovir, ganciclovir, imunovir, inosine, interferon, lamivudine, nexavir, oseltamivir, penciclovir, ribavirin, rimantadine, viramidine, zidovudine) and antifungals (Miconazole, ketoconazole, itraconazole, clotrimazole, econazole, fluconazole, voriconazole, abafungin, naftifine, caspofungin, micafungin, benzoic acid, griseofulvin).

Anti-inflammatory drugs may be, but are not limited to, zinc salts, including zinc salts of polysaccharide acids, such as hyaluronic acid, bisabolol, steroids, specifically glucocorticoids.

The filler composition optionally contains a cosmetically and/or pharmaceutically acceptable carrier. This carrier is not causing cosmetic or pharmaceutic interactions or undesired reaction, e.g. inflammation, granuloma formation. According to the instant invention the carrier may exhibit a medium in which the β-glucan moieties are suspended or solved. Particularly, said carrier may be sterile water, phosphate-buffer saline (PBS), ringer solution, isotonic saline solution (0.9%), trometamol, citrate, carbonate, acetate, borate, amino acids, diethylamine, glucono delta lactone, glycine, lactate, maleic, methanesulfonic, monoethanolamine, tartrate buffer of choice or any combination thereof. The carrier further may be selected from the group containing high purity water, glycerol, phosphate buffered saline, phosphate buffer, carbonate buffer, acetate buffer, citrate buffer, tris buffer, glycylglycine buffer or glycine buffer. Sodium phosphate buffer, sodium hydrogen phosphate buffer, sodium dihydrogen phosphate buffer, potassium phosphate buffer, potassium hydrogen phosphate buffer, potassium dihydrogen phosphate buffer or pyrophosphate buffer are preferred. Suitable are also sodium carbonate buffer, potassium carbonate buffer, sodium hydrogen carbonate buffer or potassium hydrogen carbonate buffer.

According to another embodiment of the instant invention the filler composition further comprises one or more pharmaceutical excipients. Pharmaceutical excipients are not pharmacologically active and do not exhibit volume spending or tissue augmenting property. Thus, pharmaceutical excipients may be selected from the group containing antioxidants, tonicity agents, preservatives, thickeners or mixtures thereof.

Antioxidants may be, but are not limited to, vitamin E, vitamin C, glutathione coenzyme Q, resveratrol, bisulfite sodium, butylated hydroxyl anisole/toluene, cysteinate, dithionite sodium, gentisic acid, glutamate, formaldehyde sulfoxylate sodium, metabisulfite sodium, monothiogylcerol, propyl gallate, sulfite sodium, thiogycolate sodium, flavonoids, catalase, lycopene, carotenes, lutein, superoxide dismutase and peroxidases or mixtures thereof.

Surprisingly, the optionally cross-linked β-glucan moieties, and the optional carrier, form a gel. Such gel may have thixotropic properties and/or advantageous visco-elastic properties that improve the handling, long-lasting effect, and/or syringeability of the filler composition. It was not expected by a person skilled of the art that β-glucan moieties or cross-linked β-glucan moieties form a gel that without adding other volume-augmenting substances could be used in a filler composition.

In particular embodiments, the filler composition according to the invention forms a monophasic gel.

In the context of the present invention, the term “monophasic gel” refers to a homogeneous viscoelastic cross-linked gel network comprising less than about 10%, particularly less than about 5%, less than about 2%, and most particularly less than about 1% gel particles. In a particular embodiment, the monophasic gel is free from gel particles.

In particular embodiments, the filler composition according to the invention forms gel particles. These particles are elastic and approximately spherical. Size and elastic properties depend on the parameters of the cross-linking method.

According to this aspect of the present invention, the gel particles exhibit a particle size, measured as mass median diameter by microscopical analysis or with laser diffraction, of less than or equal to about 200 μm. In a particular embodiment, the gel particle have a mass median diameter of between about 60 and 190 μm, more particularly between about 80 and 150 μm, and most particularly between about 90 and 130 μm. The particle size can be reduced by employing known techniques, such as Air jet/Air stripping method, Jet cutter method, Vortex bowl atomizer, Vibrating nozzle device, Electrostatic device, Emulsification (“water in oil”) approach, low mid and high pressure homogenization approaches. The size of the gel particle is adjusted according to the location of treatment. If the filler composition is injected the size of the gel particles provides fixation at the injection location and prevents undesirable migration to other parts of patient's body.

In one embodiment, the β-glucan moieties exhibit a mean molecular weight between about 1 kDa and about 3000 kDa. Particularly, the mean molecular weight is from about 500 kDa to about 1500 kDa. A commercially available example for such is β-glucan moiety is β-(1,3)-(1,6)-glucan from BioThera, Inc.

The β-glucan moieties are present in the filler composition for β-glucans>100 kDa at a concentration from about 0.1 wt.-% to about 5.0 wt.-% of final total weight of the filler composition. In one embodiment, the β-glucan moieties are present in the filler composition at a concentration from about 1.0 wt.-% to about 3.5 wt.-% of final total weight of the filler composition. In one further embodiment, the β-glucan moieties are present in the filler composition at a concentration from about 2.0 wt.-% to about 2.5 wt.-% of final total weight of the filler composition. The β-glucan moieties are present in the filler composition for β-glucans<=100 kDa at a concentration from about 0.1 wt.-% to about 10.0 wt.-% of final total weight of the filler composition. In one embodiment, the β-glucan moieties are present in the filler composition at a concentration from about 1.0 wt.-% to about 8.0 wt.-% of final total weight of the filler composition. In one further embodiment, the β-glucan moieties are present in the filler composition at a concentration from about 3.0 wt.-% to about 5.0 wt.-% of final total weight of the filler composition. The concentration of β-glucan moieties present in the filler composition varies according to the thixotropy/viscosity of the gel, particular or non particulated gel (size of the particles), size of the injection needle and the location of treatment.

In another aspect, the present invention relates to a method of treating a patient for aesthetic purposes, particularly by applying a dermal filler, particularly for the treatment of wrinkles and/or folds, comprising the step of administering a filler composition according to the present invention.

In certain embodiments, the filler composition of the instant invention is for use for aesthetic purposes.

In one embodiment of the present invention, the filler composition is a dermal filler.

In another embodiment, the dermal filler is for the treatment of wrinkles and/or folds.

Wrinkles that may be treated by employing the filler composition according to the instant invention include, but are not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the filler composition is for the treatment of, or for use in the treatment of, a medical condition, including lipoatrophy, gastroesophageal reflux disease (GERD), urine incontinence, vesico-ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the filler composition is for use in plastic, cosmetic, dental or general surgery, in ophthalmology, in orthopedics, for preventing tissue adhesions, or in urology.

The filler composition of the present invention may be used to produce an injectable composition for volume augmentation of tissue.

The present invention further pertains to methods of using the filler composition of the present invention for the therapeutic treatment of a patient in need thereof, such as in the treatment of lipoatrophy, gastroesophageal reflux disease (GERD), urine incontinence, vesico-ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet and perioral wrinkles.

The present invention further pertains to a method of treating a medical condition, including lipoatrophy, gastroesophageal reflux disease (GERD), urine incontinence, vesico-ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a filler composition as claimed in the present invention to a patient in need thereof.

The present invention further pertains to a method of using a filler composition according to the present invention in plastic, cosmetic, dental or general surgery, in ophthalmology, in orthopedics, as products for preventing tissue adhesions, or in urology, wherein said method comprises a step of administering a filler as claimed in the present invention to a patient in need thereof.

The present invention further pertains to a process for preparing a filler composition according to the invention, which comprises a step of adding a crosslinking reagent, particularly BDDE, to a concentrated non-crosslinked beta-glucan gel.

The present invention further pertains to a process for preparing a filler composition according to the invention, which comprises a step of combining an aqueous solution of the β-glucan moieties and a hydrophobic medium containing a water-in-oil emulsifier so as to form an emulsion of droplets of the β-glucan moieties; and adding to the emulsion a cross-linking agent capable of cross-linking the β-glucan moieties.

The present invention further pertains to a process for preparing a filler composition according to the invention, which comprises the step of combining a solution of at least one β-glucan moiety with an aqueous solution containing at least one cross-linking agent, particularly wherein:

In particular embodiments, the concentration of the β-glucan moieties used in the process of the present invention is chosen in a way that for β-glucans>100 kDa a concentration from about 0.1 wt.-% to about 5.0 wt.-% of final total weight of the filler composition is obtained, particularly a concentration from about 1.0 wt.-% to about 3.5 wt.-% of final total weight of the filler composition, and more particularly from about 2.0 wt.-% to about 2.5 wt.-% of final total weight of the filler composition. In another embodiment, the concentration of the β-glucan moieties used in the process of the present invention is chosen in a way that for β-glucans<=100 kDa a concentration from about 0.1 wt.-% to about 10.0 wt.-% of final total weight of the filler composition, particularly a concentration from about 1.0 wt.-% to about 8.0 wt.-% of final total weight of the filler composition, and more particularly from about 3.0 wt.-% to about 5.0 wt.-% of final total weight of the filler composition.

In one embodiment, the pH of the solution of the β-glucan moieties is between about pH 6 and about pH 8, more particularly between about pH 6.8 and pH 7.6.

The present invention further pertains to a kit comprising (a) the filler composition as disclosed herein, and (b) an injection device. In one embodiment, the injection device comprises a 25- to 32-gauge needle. The size of the needle will be determined by the filler composition, the depth of the injection site and the injection volume. In certain embodiments, the injection device is disposable. In one embodiment, the injection device is made of sterile glass.

The present invention further pertains to an injection device comprising a filler composition as disclosed herein. In one embodiment, the injection device may comprise a 25- to 32-gauge needle. The size of the needle will be determined by the filler composition, the depth of the injection site and the injection volume. In certain embodiments, the injection device is disposable. In one embodiment, the injection device may be made of sterile glass.

In certain embodiments, the injection device and the filler composition provided herein are both sterile and non-pyrogenic e.g. containing less than 10 EU (Endotoxin Unit, a standard measure) per dose or application. The methods of achieving the sterility of the filler composition are known to the person skilled in the art.

Isotonicity of the filler composition may be accomplished by employing sodium chloride, or other pharmaceutically acceptable agents such as dextrose.

A pharmaceutically acceptable preservative may be employed to improve the shelf-life of the filler composition. The preservative may be, but is not limited to, benzalkonium chloride, thiomersal, parabens, chlorobutanol, benzethonium chloride, m-cresol, phenol, 2-phenoxyethanol, phenyl mercuric nitrate or benzyl alcohol. The suitable concentration of the preservative agent is from about 0.001% to 5% based on the total weight of the composition and the agent selected.

The injection volume of the filler composition could be between 0.1 and 100 ml, particular between 0.1 and 50 ml, more particular between 0.1 and 30, 0.1 and 20, or 0.1 and 10 ml, and most particular between 0.1 and 5, 0.1 and 2, or 0.1 and 1 ml depending on the concentration of filler composition and the area to be treated. Alternatively, the volume can be higher than 100 ml if larger areas are augmented.

The invention is now described with reference to the following examples. These examples are provided for the purpose of illustration only and the invention should not be construed as being limited to these examples, but rather should be construed to encompass any and all variations which become evident as a result of the teaching provided herein. The following materials and methods are provided with respect to the subsequent examples but do not limit a multiplicity of materials and methodologies encompassed by the present invention.

### EXAMPLES

### Example 1

### Preparation of a Non-Particulated Beta-Glucan Gel e.g. for Use as a Dermal Filler

### Preparation of Concentrated Not Cross-Linked Beta-Glucan Gel:

Oat beta-glucan (Ceapro, Canada) or β-D-glucan from barley (Sigma-Aldrich: G6513, >95%) is dissolved or dispersed in phosphate buffer adjusted to a final pH of between pH=6 and pH=10 with a final concentration of between 5 and 20% (w/w) using a kneader.

### Conditions: 30-90 Min, Degassing of Beta-Glucan Gel

### Cross-Linking Reaction of Concentrated Beta-Glucan Gel:

BDDE (2.50% to 30% (w/w β-glucan)) is dissolved in concentrated beta-glucan gel using a kneader. After homogenization, the temperature is increased up to 40-50° C. for between 2 and 9 h. After kinetically ending the cross-linking reaction, the temperature is decreased to 20° C.

The cross-linked gel is diluted in phosphate buffer according to the finally targeted formulation.

### pH Adjustment of the Diluted Beta-Glucan Gel:

The beta-glucan gel mixture is adjusted to pH=7

Homogenization with kneader (conditions: 20° C., 15 min to 180 min).

### Ultrafiltration of the Beta-Glucan Gel:

Ultrafiltration, dialysis or chromatography of the beta-glucan gel (mixture of cross-linked and free β-glucan) in order to remove possible residues of free cross-linker and to buffer the gel mixture.

Optionally: Mixing of Cross-Linked Beta-Glucan Gel with Free Beta-Glucan:
Final Concentration of Free Beta-Glucan: 5-15% (w/v).

### Conditions: 20° C., 1 h to 10 h Until Homogeneous Distribution of Beta-Glucan Gel Mixture, Degassing of Beta-Glucan Solution

Degassing of the bulk

Sampling for determination the bioburden of the beta-glucan gel mixture

Filling of syringes with beta-glucan gel (filling of sterile syringes under aseptic conditions)

### Terminal Sterilization:

Sterilization of pre-filled syringes without plungers and backstops by steam in an autoclave at 131-133° C. using special trays made of stainless steel.

Conditions according to the results of optimization and validation.

### Visual Control and Labelling of Pre-Filled Syringes

Mounting of Pre-Filled Syringes with Backstops and Plungers

### Sealing in Blisters Together With Two Injection Needles

Final Packaging

### Example 2

### Covalent Binding of Effector Molecules to Beta-Glucan Molecules

Beta-glucan moieties can be coupled to effector molecules such as the maleinimido constructs shown in Examples 17 and 18 of WO 2007/060116 via coupling of hydroxyl groups of the β-glucan to the maleinimido Michael system.

For example, 2-(4-N,N-diethylamino-2-hydroxybenzoyl)-benzoic acid 5-N-maleinimido pentyl ester is coupled to β-glucan via at least one hydroxyl group by nucleophilic attack of the hydroxyl group to the double bond of the maleinimido group.

Beta-glucan (1-10 mg/ml) (particularly 1 mg/ml) in phosphate buffer (pH=7,4) is added to a solution of 2-(4-N,N-diethylamino-2-hydroxybenzoyl)-benzoic acid 5-N-maleinimido pentyl ester (17 mg/ml) in ethanol in a molar ratio of β-glucan:2-(4-N,N-diethylamino-2-hydroxybenzoyl)-benzoic acid 5-N-maleinimido pentyl ester of between about 1:1 to 1:2, and the mixture is shaken for 1 h at room temperature.

1-15. (canceled)

16. A filler composition comprising β-glucan moieties and, optionally, a cosmetically and/or pharmaceutically acceptable carrier, wherein the β-glucan moieties are cross-linked, and wherein the cross-linked β-glucan moieties form a monophasic gel.

17. The filler composition of claim 16, wherein the β-glucan moieties are selected from the group consisting of branched and unbranched β-glucan moieties, β-(1,3)-glucan, β-(1,4)-glucan, β-(1,3)-(1,4)-glucan, β-(1,3)-(1,6)-glucan and β-(1,4)-(1,6)-glucan moieties.

18. The filler composition of claim 16, wherein the β-glucan moieties are selected from the group consisting of cellulose, chitin, chitosan, curdlan, laminarin, chrysolaminarin, lentil pan, lichenin, plea ran, zymosan, schizophyllan, scieroglucan, β-glucans extracted from grain, and any β-glucans produced by biotechnological means.

19. The filler composition of claim 18, wherein the grain is oat or barley.

20. The filler composition of claim 16, wherein the β-glucan moieties are cross-linked by moieties that are not photo- or redox-crosslinkable.

21. The filler composition of claim 16, wherein the cross-linked β-glucan moieties are obtained by reacting β-glucan moieties with a bifunctional cross-linking agent selected from divinyl sulfone (DVS), butanediol diglycidyl ether (BDDE), 1,2,7,8-diepoxyoctane (DEO), 1,2,3,4-diepoxybutane, and polyethylene glycol diglycidyl ether, or by reacting β-glucan moieties with a multifunctional cross-linking agent.

22. The filler composition of claim 21 wherein the multifunctional cross-linking agent is polyethylene glycol epoxide.

23. The filler composition of claim 16, further comprising one or more active pharmaceutical ingredients selected from the group consisting of anesthetics, local anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents, biorejunevation agents, anti-aging agents, anti-bacterial agents, anti-histaminic agents, healing supporting agents, anti-acne agents, moisturizing agents, tanning agents and anti-cellulite agents, wherein the one or more active pharmaceutical ingredients are linked to the β-glucan moieties or a bifunctional cross-linking agent

24. The filler composition of claim 16, wherein the attendant cosmetically and/or pharmaceutically acceptable carrier is selected from the group consisting of high purity water, glycerol, phosphate buffered saline, carboxy-methylcellulose, hyaluronic acid, and mixtures thereof.

25. The filler composition of claim 16, further comprising one or more pharmaceutical excipients selected from antioxidants, viscosity enhancers/modifiers, hydrating agents, bulking substances, tonicity agents, preservatives, surface active agents, and mixtures thereof.

26. The filler composition of claim 16, wherein the concentration of the β-glucan moieties is between about 0.1 and about 5.0 wt.-% for β-glucans greater than 100 kDa based on the final total weight of filler composition, or is between about 0.1 and about 10.0 wt.-% for β-glucans less than or equal to 100 kDa based on the final total weight of filler composition.

27. The filler composition of claim 16, wherein the β-glucan moieties exhibit an average molecular weight of from about 1 kDa to about 3 mDa.

28. The filler composition of claim 16 for aesthetic purposes, as a dermal filler, for the treatment of wrinkles and/or folds, or for use in the treatment of a medical condition selected from lipoatrophy, gastroesophageal reflux disease (GERD), urine incontinence, vesico-ureteral reflux (VUR), a psychological condition caused by the appearance of an aesthetic deficiency, for use in plastic, cosmetic, dental or general surgery, in ophthalmology, in orthopedics, in preventing tissue adhesions, and in urology.

29. The filler composition of claim 16 which is an injectable filler composition.

30. A method for volume augmentation of tissue comprising injecting the filler composition of claim 16 into a human or animal subject.

31. A process for preparing the filler composition of claim 16, comprising the steps of:
(i) adding a crosslinking reagent to a concentrated non-crosslinked β-glucan gel; and
(ii) combining an aqueous solution of β-glucan moieties and a hydrophobic medium containing a water-in-oil emulsifier thereby forming an emulsion of droplets of the β-glucan moieties and adding a cross-linking agent capable of cross-linking the β-glucan moieties to the emulsion; or
(iii) combining a solution of at least one β-glucan moiety with an aqueous solution containing at least one cross-linking agent, wherein:
a. the pH of the aqueous solution containing at least one cross-linking agent is adjusted to a pH value of between about 6 and about 10;
b. the concentration of the cross-linking agent is between about 2.5 wt-% (w/w β-glucan) and 30 wt-% (w/w β-glucan), or between about 2.5 wt-% (w/w β-glucan) and 15 wt-% (w/w β-glucan);
c. the concentration of β-glucan moieties is between about 0.1 and about 5.0 wt-% for β-glucans greater than 100 kDa based on the final total weight of filler composition, or between about 0.1 and about 10.0 wt.-% for β-glucans less than or equal to 100 kDa based on the final total weight of filler composition;
d. the viscosity of the solution of β-glucan moieties is adjusted to a force of between about 15 N and about 50 N for pressing the solution of β-glucan moieties through a 27G1/2 needle; and/or
e. the aqueous solution containing the cross-linking agent optionally further comprises one or more active pharmaceutical ingredients selected from the group consisting of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

32. The process of claim 31, herein the crosslinking agent is BODE.

33. A kit comprising the filler composition of claim 29 and an injection device, wherein the injection device is a syringe or an electronic injection device.

34. An injection device comprising the filler composition of claim 29.
